(19) United States
(12) Patent Application Publication
(10) Pub. No.: US 2024/0358972 A1
(43) Pub. Date: Oct. 31, 2024

(54) Intravenous Injection Site Stabilization Device With Switchable Adhesive

(71) Applicant: Becton, Dickinson and Company, Franklin Lakes, NJ (US)

(72) Inventors: Linjie Zhu, Livingston, NJ (US); James Joseph Semler, Randolph, NJ (US)

(21) Appl. No.: 18/687,844

(22) PCT Filed: Aug. 31, 2022
(86) PCT No.: PCT/US22/42144

§ 371 Date: Feb. 29, 2024

Related U.S. Application Data
(60) Provisional application No. 63/240,025, filed on Sep. 2, 2021.

(51) Int. Cl. A61M 25/02

(52) U.S. Cl. CPC A61M 25/02; A61M 2025/0266; A61M 2025/028

(57) ABSTRACT

An intravenous (IV) injection site stabilization device that includes a securement device and an adhesive pad comprising a top layer and a bottom layer, where the securement device is attached to the top layer and wherein the bottom layer comprises a switchable adhesive, where the switchable adhesive is configured to adhere to a skin of a user, and where the switchable adhesive has an adhering state and a removal state, such that a first amount of force with which the switchable adhesive adheres to a surface in the adhering state is greater than a second amount of force with which the switchable adhesive adheres to a surface in the removal state.

### CROSS-REFERENCE TO RELATED APPLICATION

The present application claims priority to U.S. Provisional Application Ser. No. 63/240,025, entitled “Intravenous Injection Site Stabilization Device with Switchable Adhesive”, filed Sep. 2, 2021, the entire disclosure of which is hereby by incorporated by reference in its' entirety.

### BACKGROUND

Intravenous (IV) therapy may refer to a medical technique that is used to deliver fluids, medications, and/or nutrition directly into a body of a patient via an IV line (e.g., an IV cannula, an IV catheter, etc.) inserted into a vein. IV therapy may be used for rehydration, to provide nutrition to a person who cannot consume food or water by mouth, and/or to administer medications or other medical therapy, such as blood products or electrolytes.

In some instances, the IV line may include the use of a securement device that uses a backing material with an adhesive, such as tape, that is attached to the skin of the patient at or near an IV injection site to hold the IV line in place. Various adhesives may be used. Hydrocolloid and/or zinc oxide-based adhesives have less of a tendency to excoriate the skin of a patient when removed and that tendency may be particularly important for a patient whose skin is more sensitive or fragile. Hydrocolloid and/or zinc oxide-based adhesives may provide a low adhesion force between the adhesive and the skin, which allows minimal skin injury during a process of removing the adhesive from the skin.

However, it may also be important to minimize movement of components of the IV line, such as an IV catheter. In some instances, if the IV catheter is not properly secured in place, the IV catheter may be displaced from an intended location. Further, repeated movement of the IV catheter, to correctly position the IV catheter, can cause irritation of a blood vessel, disrupt proper introduction of medications to the patient, and/or increase the potential for bleeding or infection at the IV insertion site. If extensive movement occurs, the IV catheter could even come out of the patient, interrupting delivery of medication and requiring re-insertion, potentially with a requirement for hospitalization of the patient. Accordingly, a strong adhesion force between the adhesive and the skin may be desired to ensure secure positioning of the IV catheter during usage.

Therefore, there is an increasing need for a device that holds an IV line and/or components of the IV line securely in place during use of the IV line and that allows for removal of the IV line with minimal injury to the patient.

### SUMMARY

Accordingly, provided are improved systems, devices, products, apparatus, and/or methods for an IV injection site stabilization device with a switchable adhesive.

Clause 1: An intravenous (IV) injection site stabilization device having a securement device, and an adhesive pad having a top layer and a bottom layer, wherein the securement device is attached to the top layer and wherein the bottom layer comprises a switchable adhesive. The switchable adhesive is configured to adhere to a skin of a user, and the switchable adhesive has an adhering state and a removal state. Wherein a first amount of force with which the switchable adhesive adheres to a surface in the adhering state is greater than a second amount of force with which the switchable adhesive adheres to a surface in the removal state.

Clause 2: The IV injection site stabilization device of Clause 1, wherein the switchable adhesive is configured to transition between the adhering state and the removal state based on the switching adhesive being exposed to an external stimulus.

Clause 3: The IV injection site stabilization device of Clause 2, wherein the external stimulus includes at least one of the following: light, a solvent, temperature, a substance with a predetermined pH level, moisture, electricity, or any combination thereof.

Clause 4: The IV injection site stabilization device of Clause 1, wherein the switchable adhesive comprises a light switchable adhesive, wherein the light switchable adhesive is configured to transition between the adhering state and the removal state based on the light switchable adhesive being exposed to light.

Clause 5: The IV injection site stabilization device of Clause 4, wherein the light switchable adhesive is configured to transition between the adhering state and the removal state based on the light switchable adhesive being exposed to ultraviolet (UV) light.

Clause 6: The IV injection site stabilization device of Clause 1, wherein the securement device includes a transparent material.

Clause 7: The IV injection site stabilization device of Clause 1, wherein the top layer of the adhesive pad includes a transparent material.

Clause 8: The IV injection site stabilization device of Clause 1, wherein the top layer of the adhesive pad includes a liquid permeable material.

Clause 9: The IV injection site stabilization device of Clause 1, further including a liner, wherein the bottom layer of the adhesive pad has a bottom surface, and wherein the liner is positioned on the bottom surface of the adhesive pad.

Clause 10: The IV injection site stabilization device of Clause 1, wherein the top layer of the adhesive pad includes a plastic material, a paper material, a non-woven material, a foam material, or any combination thereof.

Clause 11: The IV injection site stabilization device of Clause 1, further including a protective film that is opaque, wherein the protective film is positioned to cover the securement device and the top layer of the adhesive pad.

Clause 12: The IV injection site stabilization device of Clause 1, wherein the securement device includes a catheter retainer.

Clause 13: An intravenous (IV) injection site stabilization device including a securement device, an adhesive pad including a top layer and a bottom layer, wherein the securement device is attached to the top layer and wherein the bottom layer includes a light switchable adhesive. The device also includes a protective film that includes an opaque material, wherein the protective film is positioned to cover a top surface of the securement device and a top surface of the adhesive pad, wherein the light switchable adhesive is configured to adhere to a layer of skin of a user, and wherein the light switchable adhesive has an adhering state and a removal state, wherein a first amount of force with which the light switchable adhesive adheres to a surface in the adhering state is greater than a second amount of force with which the light switchable adhesive adheres to a surface in the removal state.

Clause 14: The IV injection site stabilization device of Clause 13, further including a liner, wherein the bottom layer of the adhesive pad has a bottom surface and wherein the liner is positioned on the bottom surface of the adhesive pad.

Clause 15: The IV injection site stabilization device of Clause 13, wherein the light switchable adhesive is configured to transition between the adhering state and the removal state based on the light switchable adhesive being exposed to ultraviolet light and/or visible light.

Clause 16: The IV injection site stabilization device of Clause 13, wherein the securement device includes a transparent material and wherein the top layer of the adhesive pad comprises a transparent material.

Clause 17: An intravenous (IV) injection site stabilization device including a securement device, and an adhesive pad having a top layer and a bottom layer, wherein the securement device is attached to the top layer and wherein the bottom layer includes a light switchable adhesive. The light switchable adhesive is configured to adhere to a layer of skin of a user; and the light switchable adhesive has an adhering state and a removal state, wherein a first amount of force with which the light switchable adhesive adheres to a surface in the adhering state is greater than a second amount of force with which the light switchable adhesive adheres to a surface in the removal state.

Clause 18: The IV injection site stabilization device of Clause 17, further including a protective film that is opaque, wherein the protective film is positioned to cover a top surface of the securement device and a top surface of the adhesive pad, and a liner, wherein the bottom layer of the adhesive pad has a bottom surface; and wherein the liner is positioned on the bottom surface of the adhesive pad.

Clause 19: The IV injection site stabilization device of Clause 17, wherein the light switchable adhesive is configured to transition between the adhering state and the removal state based on the light switchable adhesive being exposed to ultraviolet light and/or visible light.

Clause 20: The IV injection site stabilization device of Clause 17, wherein the securement device includes a transparent material and wherein the top layer of the adhesive pad includes a transparent material.

Clause 21: An intravenous (IV) injection site stabilization device including a securement device, and an adhesive pad including a top layer and a bottom layer, wherein the securement device is attached to the top layer and wherein the bottom layer includes a liquid switchable adhesive. The liquid switchable adhesive is configured to adhere to a layer of skin of a user, and the liquid switchable adhesive has an adhering state and a removal state, wherein a first amount of force with which the liquid switchable adhesive adheres to a surface in the adhering state is greater than a second amount of force with which the liquid switchable adhesive adheres to a surface in the removal state.

Clause 22: The IV injection site stabilization device of Clause 21, further including a protective film that is not liquid permeable, wherein the protective film is positioned to cover a top surface of the securement device and a top surface of the adhesive pad, and a liner. The bottom layer of the adhesive pad has a bottom surface, and the liner is positioned on the bottom surface of the adhesive pad.

Clause 23: The IV injection site stabilization device of Clause 21, wherein the liquid switchable adhesive is configured to transition between the adhering state and the removal state based on the liquid switchable adhesive being exposed to a predetermined liquid.

Clause 24: The IV injection site stabilization device of Clause 21, wherein the securement device includes a liquid permeable material and wherein the top layer of the adhesive pad includes a liquid permeable material.

Clause 25: The IV injection site stabilization device of Clause 21, wherein the top layer of the adhesive pad includes a liquid permeable material.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of a non-limiting embodiment of an IV injection site stabilization device;

FIG. 2 is a diagram of the IV injection site stabilization device shown in FIG. 1 attached to the skin of a user;

FIG. 3 is a diagram of the IV injection site stabilization device shown in FIG. 1 with a protective film;

FIG. 4 is a diagram of an exploded view of a non-limiting embodiment of an IV injection site stabilization device; and

FIG. 5 is a diagram of an assembled view of the IV injection site stabilization device shown in FIG. 4.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided are improved devices, systems, methods, and products for an IV injection site stabilization device. Embodiments of the present disclosure may include IV injection site stabilization device that includes a securement device and an adhesive pad comprising a top layer and a bottom layer, where the securement device is attached to the top layer and wherein the bottom layer comprises a switchable adhesive, where the switchable adhesive is configured to adhere to a layer of skin of a user and where the switchable adhesive has an adhering state and a removal state, such that a first amount of force with which the switchable adhesive adheres to a surface in the adhering state is greater than a second amount of force with which the switchable adhesive adheres to a surface in the removal state. In some non-limiting embodiments, the switchable adhesive is configured to transition between the adhering state and the removal state based on the switching adhesive being exposed to an external stimulus. In some non-limiting embodiments, the external stimulus comprises at least one of the following: light, a solvent, temperature, a substance with a predetermined pH level, moisture, electricity, or any combination thereof. In some non-limiting embodiments, the switchable adhesive includes a light switchable adhesive, wherein the light switchable adhesive is configured to transition between the adhering state and the removal state based on the light switchable adhesive being exposed to light. In some non-limiting embodiments, the light switchable adhesive is configured to transition between the adhering state and the removal state based on the light switchable adhesive being exposed to ultraviolet (UV) light. In some non-limiting embodiments, the securement device includes a transparent material. In some non-limiting embodiments, the top layer of the adhesive pad includes a transparent material. In some non-limiting embodiments, the top layer of the adhesive pad includes a liquid permeable material. In some non-limiting embodiments, the IV injection site stabilization device further comprises a liner, where the bottom layer of the adhesive pad has a bottom surface and where the liner is positioned on the bottom surface of the adhesive pad. In some non-limiting embodiments, the top layer of the adhesive pad includes a plastic material, a paper material, a non-woven material, a foam material, or any combination thereof. In some non-limiting embodiments, the IV injection site stabilization device further comprises a protective film that is opaque, where the protective film is positioned to cover the securement device and the top layer of the adhesive pad. In some non-limiting embodiments, the securement device comprises a catheter retainer.

In this way, embodiments of the present disclosure allow for an IV injection site stabilization device that holds an IV line and/or components of the IV line securely in place during use of the IV line on a patient and that allows for removal of the IV injection site stabilization device and/or the IV line with minimal injury to the patient once the IV line is to be repositioned or is no longer necessary.

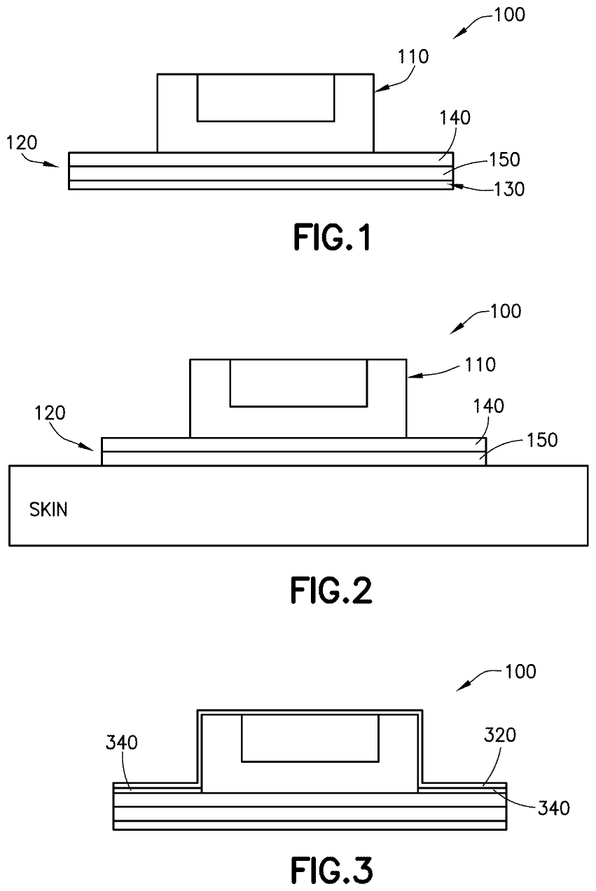

Referring now to FIGS. 1-2, FIGS. 1-2 are diagrams of intravenous (IV) injection site stabilization device 100 and components thereof. As shown in FIGS. 1-2, IV injection site stabilization device 100 may include securement device 110, adhesive pad 120, and liner 130. Adhesive pad 120 may include top layer 140 and bottom layer 150, where a bottom surface of top layer 140 is positioned on a top surface of bottom layer 150. In some non-limiting embodiments, bottom layer 150 may include a switchable adhesive that is configured to adhere to a layer of skin of a user (e.g., a patient). For example, bottom layer 150 may include a switchable adhesive as a coating on a bottom surface of bottom layer 150. In another example, bottom layer 150 may be the switchable adhesive, such that the switchable adhesive is a coating on a bottom surface of top layer 140. The thickness of adhesive pad is usually not exceeding 3 mm, but depends on the material of the top layer. The bottom layer is normally below 200 micrometers. The height of the securement mechanism can vary, but typically below 0.5 inch. In some non-limiting embodiments, top layer 140 may be sized and configured to cover a same amount of surface area as bottom layer 150. In some non-limiting embodiments, top layer 140 may be sized and configured to cover a different amount of surface area as bottom layer 150. For example, top layer 140 may be larger or smaller than bottom layer 150 with regard to an amount of surface area covered by top layer 140.

In some non-limiting embodiments, adhesive pad 120 may include a plurality of apertures that is sized and configured to allow for one or more sutures to be used to attach IV injection site stabilization device 100 to a surface (e.g., skin of a user). In some non-limiting embodiments, liner 130 may be made of a plurality of pieces. For example, liner 130 may be made of two separate pieces. In this way, liner 130 may be more easily removed than if liner 130 is made of a single piece. In addition, liner 130 may allow for easier placement of IV injection site stabilization device 100 based on liner 130 being made of two separate pieces.

In some non-limiting embodiments, securement device 110 may be positioned on top layer 140. For example, securement device 110 may be attached to top layer 140 via a mechanical fixture (e.g., using a fastener), an adhesive, a welding process, and/or the like. In some non-limiting embodiments, securement device 110 may include a retaining mechanism (e.g., a retainer, a lock, a clip, a hook, a clamp, an anchor, etc.) for holding a component of an IV line. For example, securement device 110 may include a catheter retainer.

In some non-limiting embodiments, liner 130 may be positioned on bottom layer 150. For example, liner 130 may be positioned on the bottom surface of bottom layer 150. In some non-limiting embodiments, liner 130 may include a releasable liner that is sized and configured to cover the switchable adhesive on bottom layer 150 and to be removed prior to or during use of IV injection site stabilization device 100. As shown in FIG. 2, when liner 130 is removed, the switchable adhesive of bottom layer 150 may adhere to the skin of the user. In this way, liner 130 may prevent unintentional adherence of IV injection site stabilization device 100 and may prevent the adherence of foreign objects to the switchable adhesive before IV injection site stabilization device 100 is applied to a surface.

In some non-limiting embodiments, the switchable adhesive has an adhering state and a removal state. In some non-limiting embodiments, an amount of force with which the switchable adhesive adheres to a surface (e.g., an amount of force that is required to remove the switchable adhesive from a surface) in the adhering state is greater than an amount of force with which the switchable adhesive adheres to a surface in the removal state. For example, the amount of force with which the switchable adhesive adheres to a surface in the removal state may be at least 30% less than the amount of force with which the switchable adhesive adheres to a surface in the adhering state.

In some non-limiting embodiments, the switchable adhesive may be configured to transition between the adhering state and the removal state based on (e.g., in response to, according to the triggering condition of, etc.) the switching adhesive being exposed to an external stimulus (e.g., only an external stimulus and no other stimulus). For example, the switchable adhesive may transition between the adhering state and the removal state based on the switching adhesive being exposed to electromagnetic radiation (e.g., light, such as ultraviolet (UV) light, visible light, etc.), a solvent (e.g., alcohol, such as isopropyl alcohol), a temperature (e.g., a threshold temperature, a threshold of change in temperature, etc.), a substance with a predetermined pH level (e.g., a substance with a predetermined pH level within a threshold range of pH level), moisture, electricity (e.g., an electrical current, a voltage, etc.), or a combination thereof. Examples of switchable adhesives may be found in U.S. Pat. Nos. 8,043,699, 8,697,932, 9,040,076, and U.S. Patent Application Publication No. 2018/0030321, each of which are incorporated herein by reference in their entireties. In some non-limiting embodiments, the switchable adhesive may be configured to transition between the adhering state and the removal state a number of times (e.g., a number of cycles from the adhering state and the removal state) as appropriate for an application.

In some non-limiting embodiments, the switchable adhesive may include a light switchable adhesive that is configured to transition between the adhering state and the removal state based on exposure of the light switchable adhesive to light (e.g., electromagnetic radiation having a predetermined wavelength). For example, the switchable adhesive may include a light switchable adhesive that is configured to transition between the adhering state and the removal state based on the light switchable adhesive being exposed to light having a predetermined wavelength. In some non-limiting embodiments, the light switchable adhesive is configured to transition between the adhering state and the removal state based on the light switchable adhesive being exposed to ultraviolet light (e.g., electromagnetic radiation having a wavelength in a range between 100-400 nm). In some non-limiting embodiments, the light switchable adhesive is configured to transition between the adhering state and the removal state based on being exposed to light having a wavelength in a range between 100-280 nm (e.g., in the UVC spectrum), light having a wavelength in a range between 280-315 nm (e.g., in the UVB spectrum), and/or light having a wavelength in a range between 315-400 nm (e.g., in the UVA spectrum).

In some non-limiting embodiments, the switchable adhesive is configured to transition between the adhering state and the removal state being exposed to a liquid. For example, the switchable adhesive is configured to transition from the adhering state to the removal state being exposed to a liquid with specific chemical properties, which may be referred to as a deactivation liquid, or to transition from the removal state to the adhering state based on the liquid switchable adhesive being exposed to a liquid with specific chemical properties, which may be referred to as an activation liquid. The liquid may also be safe for exposure to skin of a user. In one example, a deactivation liquid may include a combination of water and at least one non-aqueous solvent. In another example, an activation liquid may include a combination of water, at least one non-aqueous solvent, and a plasticizer. In some non-limiting embodiments, the at least one non-aqueous solvent may include acetone, acetonitrile, methanol, ethanol, isopropyl alcohol, n-propanol, n-butanol, 2-butanol, isobutanol, 2-methy-2-butanol, n-pentanol, n-hexanol, 2-hexanol, cyclohexanol, n-heptanol, noctanol, n-nonanol, n-decanol, undecanol, dodecanol, 1-tetradecanol, propylene glycol, ethylene glycol, butylene glycol, arachidyl alcohol, docosanol, tetracosanol, hexacosanol, octanosol, triacontanol, cetyl alcohol, stearyl alcohol, polycosinol, methyl ethyl ketone, ethyl acetate, mineral spirits, linseed oil, vegetable oil, citrus based solvents, limonene, and any combinations thereof.

In some non-limiting embodiments, securement device 110 and/or top layer 140 may include a transparent material. For example, securement device 110 and/or top layer 140 may be made from (e.g., constructed of) a transparent material. In this way, light may be able to travel through securement device 110 and/or top layer 140 and reach a light switchable adhesive of bottom layer 150 (e.g., a light switchable adhesive on a bottom surface of bottom layer 150) to cause the light switchable adhesive to transition from the adhering state and the removal state or to transition from the removal state and the adhering state. In some non-limiting embodiments, securement device 110 and/or top layer 140 may include a transparent material that is transparent to light having a first wavelength (e.g., light having a predetermined wavelength, light having a predetermined range of wavelengths, etc.) and/or that is opaque to light having a second wavelength (e.g., which is different than the first wavelength).

In some non-limiting embodiments, securement device 110 and/or top layer 140 may include a liquid permeable material. For example, securement device 110 and/or top layer 140 may be made from (e.g., constructed of) a liquid permeable material. In this way, a liquid may be able to travel through securement device 110 and/or top layer 140 and reach a liquid switchable adhesive of bottom layer 150 (e.g., a liquid switchable adhesive on a bottom surface of bottom layer 150) to cause the liquid switchable adhesive to transition between the adhering state and the removal state. In some non-limiting embodiments, securement device 110 and/or top layer 140 may include a liquid permeable material that is permeable to a first liquid having first chemical properties (e.g., a liquid having a specific molecular size, a liquid having a specific chemical composition, etc.) and/or that is impermeable to a second liquid having second chemical properties (e.g., which is different than the first chemical properties).

In some non-limiting embodiments, top layer 140 and bottom layer 150 of adhesive pad 120 may be constructed by a co-extrusion process and/or a lamination process. In some non-limiting embodiments, top layer 140 of adhesive pad 120 may include (e.g., be made from) a plastic material, a paper material, a non-woven material, a foam material, or any combination thereof. In some non-limiting embodiments, top layer 140 may include a flexible material, such as a flexible elastomer (e.g., a flexible transparent elastomer). In this way, top layer 140 may provide a degree of comfort when applied to the skin of a user. In some non-limiting embodiments, top layer 140 may include a plurality of layers. For example, top layer 140 may be made from an upper layer that includes a rigid material (e.g., a rigid elastomer and/or a rigid polymer, such as polyester) and a lower layer that includes a flexible material (e.g., a flexible elastomer).

Referring now to FIG. 3, FIG. 3 is a diagram of IV injection site stabilization device 100 with protective film 320. As shown in FIG. 3, protective film 320 may be positioned on top of securement device 110 and/or top layer 140 to cover a top surface of securement device 110 and a top surface of adhesive pad 120. As further shown in FIG. 3, protective film 320 may be held in place with adhesive layer 340. In some non-limiting embodiments, adhesive layer 340 may include an adhesive (e.g., a pressure sensitive adhesive, a switchable adhesive, etc.) that holds protective film 320 in place on top of securement device 110 and/or top layer 140 of adhesive pad 120. In some non-limiting embodiments, protective film 320 may be held in place without adhesive layer 340. For example, adhesive layer 340 may not be included, and protective film 320 may be held in place based on tension forces, static forces, friction forces, and/or the like.

In some non-limiting embodiments, protective film 320 may be positioned to cover a portion of or all of securement device 110 and/or top layer 140 of adhesive pad 120. In some non-limiting embodiments, protective film 320 may be made from an opaque material. In this way, protective film 320 may prevent light from travelling through securement device 110 and/or top layer 140 to reach a light switchable adhesive of bottom layer 150 (e.g., a light switchable adhesive on a bottom surface of bottom layer 150), which may prevent the light switchable adhesive from transitioning between the adhering state and the removal state. In some non-limiting embodiments, protective film 320 may be made from a liquid impermeable material. In this way, protective film 320 may prevent a liquid from travelling through securement device 110 and/or top layer 140 to reach a liquid switchable adhesive of bottom layer 150 (e.g., a liquid switchable adhesive on a bottom surface of bottom layer 150), which may prevent the liquid switchable adhesive from transitioning between the adhering state and the removal state.

In some non-limiting embodiments, protective film 320 may be positioned on top of only one of securement device 110 or top layer 140. For example, where top layer 140 is made from a transparent material but securement device 110 is not made from a transparent material, protective film 320 may be positioned on top of top layer 140 and not on top of securement device 110. In another example, where securement device 110 is made from a transparent material but top layer 140 is not made from a transparent material, protective film 320 may be positioned on top of securement device 110 and not on top of top layer 140.

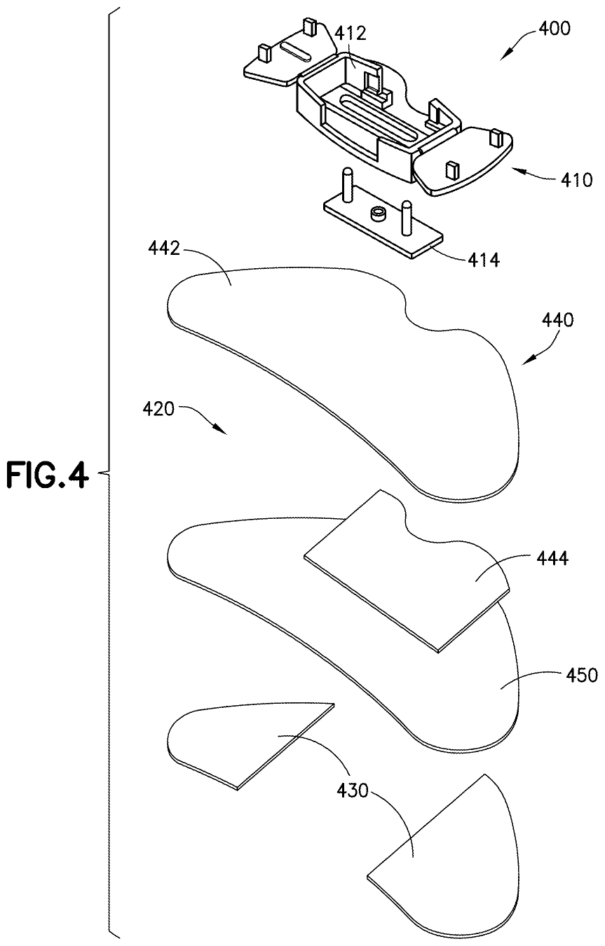

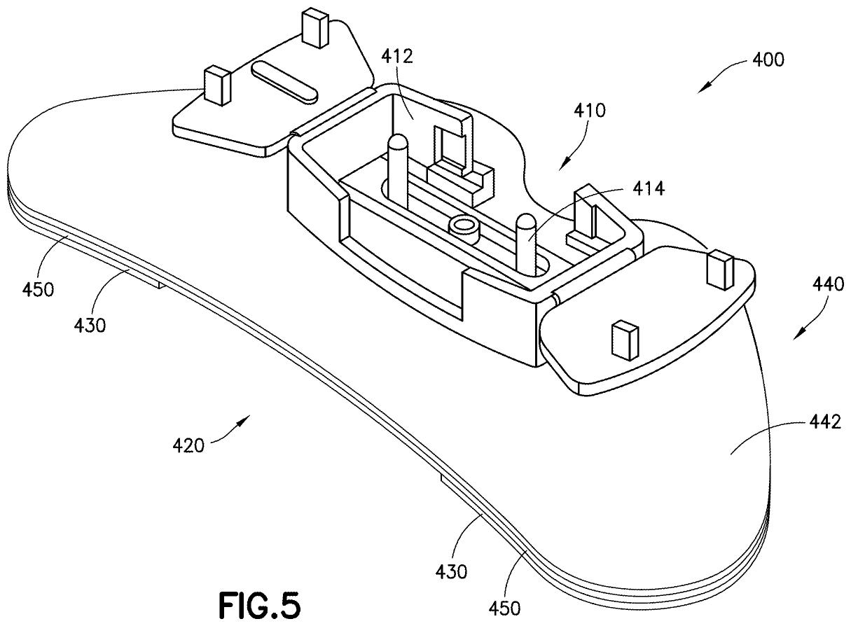

Referring now to FIGS. 4 and 5, FIGS. 4-5 are diagrams of IV injection site stabilization device 400 and components thereof. As shown in FIGS. 4-5, IV injection site stabilization device 400 may include securement device 410, adhesive pad 420, and liner 430. In some non-limiting embodiments, securement device 410 may be the same as or similar to secure device 110, adhesive pad 420 may be the same as or similar to adhesive pad 120, and liner 430 may be the same as or similar to liner 130. Adhesive pad 420 may include top layer 440 and bottom layer 450, where a bottom surface of top layer 440 is positioned on a top surface of bottom layer 450. In some non-limiting embodiments, top layer 440 may be the same as similar to top layer 140 and bottom layer 450 may be the same as or similar to bottom layer 150.

As shown in FIG. 4, securement device 410 may include retaining mechanism 412 for holding a component of an IV line and base section 414 for attaching retaining mechanism 412 to top layer 440. In some non-limiting embodiments, retaining mechanism 412 may include a catheter retainer. As further shown in FIG. 4, top layer 440 may include upper layer 442 and lower layer 444. In some non-limiting embodiments, upper layer 442 may include a rigid material (e.g., a rigid elastomer and/or a rigid polymer, such as polyester). In some non-limiting embodiments, lower layer 444 may include a flexible material (e.g., a flexible elastomer). In some non-limiting embodiments, upper layer 442 and lower layer 444 may be made from a transparent material. In some non-limiting embodiments, upper layer 442 may be sized and configured to cover a same amount of surface area as lower layer 444. In some non-limiting embodiments, upper layer 442 may be sized and configured to cover a different amount of surface area as lower layer 444. For example, upper layer 442 may be larger or smaller than lower layer 444 with regard to an amount of surface area covered by upper layer 442. As further shown in FIG. 4, liner 430 may be made of two pieces.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. An intravenous (IV) injection site stabilization device comprising:
a securement device; and
an adhesive pad comprising a top layer and a bottom layer, wherein the securement device is attached to the top layer and wherein the bottom layer comprises a switchable adhesive;
wherein the switchable adhesive is configured to adhere to a skin of a user;
wherein the switchable adhesive has an adhering state and a removal state, and wherein a first amount of force with which the switchable adhesive adheres to a surface in the adhering state is greater than a second amount of force with which the switchable adhesive adheres to a surface in the removal state.

2. The IV injection site stabilization device of claim 1, wherein the switchable adhesive is configured to transition between the adhering state and the removal state based on the switching adhesive being exposed to an external stimulus.

3. The IV injection site stabilization device of claim 2, wherein the external stimulus comprises at least one of the following:
light;
a solvent;
temperature;
a substance with a predetermined pH level;
moisture;
electricity; or
or any combination thereof.

4. The IV injection site stabilization device of claim 1, wherein the switchable adhesive comprises a light switchable adhesive, wherein the light switchable adhesive is configured to transition between the adhering state and the removal state based on the light switchable adhesive being exposed to light.

5. The IV injection site stabilization device of claim 4, wherein the light switchable adhesive is configured to transition between the adhering state and the removal state based on the light switchable adhesive being exposed to ultraviolet (UV) light.

6. The IV injection site stabilization device of claim 1, wherein the securement device comprises a transparent material.

7. The IV injection site stabilization device of claim 1, wherein the top layer of the adhesive pad comprises a transparent material.

8. The IV injection site stabilization device of claim 1, wherein the top layer of the adhesive pad comprises a liquid permeable material.

9. The IV injection site stabilization device of claim 1, further comprising:
a liner;
wherein the bottom layer of the adhesive pad has a bottom surface; and
wherein the liner is positioned on the bottom surface of the adhesive pad.

10. The IV injection site stabilization device of claim 1, wherein the top layer of the adhesive pad comprises a plastic material, a paper material, a non-woven material, a foam material, or any combination thereof.

11. The IV injection site stabilization device of claim 1, further comprising:
a protective film that is opaque;
wherein the protective film is positioned to cover the securement device and the top layer of the adhesive pad.

12. The IV injection site stabilization device of claim 1, wherein the securement device comprises a catheter retainer.

13. An intravenous (IV) injection site stabilization device comprising:
a securement device;
an adhesive pad comprising a top layer and a bottom layer, wherein the securement device is attached to the top layer and wherein the bottom layer comprises a light switchable adhesive; and
a protective film that comprises an opaque material, wherein the protective film is positioned to cover a top surface of the securement device and a top surface of the adhesive pad;
wherein the light switchable adhesive is configured to adhere to a layer of skin of a user; and
wherein the light switchable adhesive has an adhering state and a removal state, wherein a first amount of force with which the light switchable adhesive adheres to a surface in the adhering state is greater than a second amount of force with which the light switchable adhesive adheres to a surface in the removal state.

14. The IV injection site stabilization device of claim 13, further comprising:
a liner;
wherein the bottom layer of the adhesive pad has a bottom surface; and
wherein the liner is positioned on the bottom surface of the adhesive pad.

15. The IV injection site stabilization device of claim 13, wherein the light switchable adhesive is configured to transition between the adhering state and the removal state based on the light switchable adhesive being exposed to ultraviolet light or visible light.

16. The IV injection site stabilization device of claim 13, wherein the securement device comprises a transparent material and wherein the top layer of the adhesive pad comprises a transparent material.

17. An intravenous (IV) injection site stabilization device comprising:
a securement device; and
an adhesive pad comprising a top layer and a bottom layer, wherein the securement device is attached to the top layer and wherein the bottom layer comprises a light switchable adhesive;
wherein the light switchable adhesive is configured to adhere to a layer of skin of a user; and
wherein the light switchable adhesive has an adhering state and a removal state, wherein a first amount of force with which the light switchable adhesive adheres to a surface in the adhering state is greater than a second amount of force with which the light switchable adhesive adheres to a surface in the removal state.

18. The IV injection site stabilization device of claim 17, further comprising:
a protective film that is opaque, wherein the protective film is positioned to cover a top surface of the securement device and a top surface of the adhesive pad; and
a liner;
wherein the bottom layer of the adhesive pad has a bottom surface; and
wherein the liner is positioned on the bottom surface of the adhesive pad.

19. The IV injection site stabilization device of claim 17, wherein the light switchable adhesive is configured to transition between the adhering state and the removal state based on the light switchable adhesive being exposed to ultraviolet light or visible light.

20. The IV injection site stabilization device of claim 17, wherein the securement device comprises a transparent material and wherein the top layer of the adhesive pad comprises a transparent material.

21. An intravenous (IV) injection site stabilization device comprising:
a securement device; and
an adhesive pad comprising a top layer and a bottom layer, wherein the securement device is attached to the top layer and wherein the bottom layer comprises a liquid switchable adhesive;
wherein the liquid switchable adhesive is configured to adhere to a layer of skin of a user; and
wherein the liquid switchable adhesive has an adhering state and a removal state, wherein a first amount of force with which the liquid switchable adhesive adheres to a surface in the adhering state is greater than a second amount of force with which the liquid switchable adhesive adheres to a surface in the removal state.

22. The IV injection site stabilization device of claim 21, further comprising:
a protective film that is not liquid permeable, wherein the protective film is positioned to cover a top surface of the securement device and a top surface of the adhesive pad; and
a liner;
wherein the bottom layer of the adhesive pad has a bottom surface; and
wherein the liner is positioned on the bottom surface of the adhesive pad.

23. The IV injection site stabilization device of claim 21, wherein the liquid switchable adhesive is configured to transition between the adhering state and the removal state based on the liquid switchable adhesive being exposed to a predetermined liquid.

24. The IV injection site stabilization device of claim 21, wherein the securement device comprises a liquid permeable material and wherein the top layer of the adhesive pad comprises a liquid permeable material.

25. The IV injection site stabilization device of claim 21, wherein the top layer of the adhesive pad comprises a liquid permeable material.
